(19) United States
(12) Patent Application Publication
(10) Pub. No.: US 2010/0227935 A1
(43) Pub. Date: Sep. 9, 2010

(54) CLASS OF TERPENE-DERIVED COMPOUNDS HAVING AN ANTIBIOTIC ACTIVITY, COMPOSITIONS CONTAINING THE SAME AND USES THEREOF

(75) Inventors: Jean-Paul Leonetti, Castelnau-Le-Lez (FR); Maxime Gualtieri, Gallargues Le Montueux (FR); Laurence Coulibeuf, Marseille (FR); Gaëtan Herbette, Marseille (FR)

Correspondence Address: BIRCH STEWART KOLASCH & BIRCH, PO BOX 747, FALLS CHURCH, VA, 22040-0747, US

(73) Assignees: Centre National De La Recherche Scientifique, Paris (FR); Universite De Provence-Aix-Marseille 1, Marseille (FR); Universite Paul Cezanne-Aix-Marseille 3, Aix-En-Provence (FR)

(21) Appl. No.: 12/442,468

(22) PCT Filed: Sep. 21, 2007
(86) PCT No.: PCT/FR2007/001544

§ 371 Date: Apr. 30, 2010

(30) Foreign Application Priority Data
Sep. 22, 2006 (FR) 0608345
Mar. 30, 2007 (FR) 0702357

(51) Int. Cl. A61K 31/121; C07C 49/255; C07C 225/14; C07C 321/00; C07C 49/203; C12P 13/00; C12P 11/00; C12P 7/26; A61K 31/131; C12N 1/20; A61P 31/04

(52) U.S. Cl. 514/666; 568/413; 564/502; 568/42; 568/412; 435/128; 435/130; 435/148; 514/675; 435/252.1

(57) ABSTRACT

The present invention relates to a new class of terpene-derived compounds having an antibiotic activity and of the formulae (I) or (II) in which A is selected from NR₁, O, S, CR₂R₃ or R—(CH₂)ₙ—R′, wherein R and R′ represent independently NH, O, S or CH₂, and in which R₁, R₂ et R₃ are substituents and n is higher than or equal to 1, in particular equal to 2. The invention relates to the compound according to both the formula (I) or the formula (II) taken individually, to mixtures of the two compounds, and to compositions, mainly therapeutic ones, comprising at least one of the compounds or said mixture. According to a particular embodiment, the compound ((I) or (II) taken individually, the mixtures of both compounds and the compositions of the invention are used in the treatment of difficult C-related digestive infections. In one particular embodiment, the compound of the formula (I) is margaucine, a compound produced by a bacterial stem.

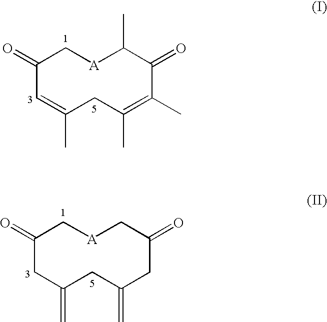

The present application relates to a novel class of compounds derived from terpene, having antibiotic activity, with formulae (I) or (II):

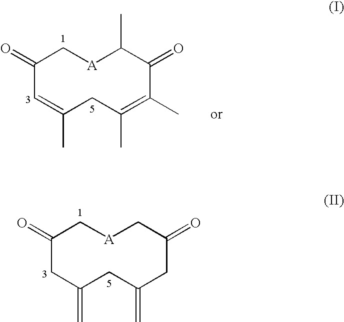

in which A is selected from NR₁, O, S, CR₂R₃ and R—(CH₂)ₙ—R′ in which R and R′, independently of each other, are NH, O, S or CH₂ and R₁, R₂, R₃ are substituents, and n is equal to 1 or more, in particular equal to 2.

### BACKGROUND OF THE INVENTION

Doubt is now being cast on advances brought about by antibiotics in the medical field because of the appearance and propagation of germs which are resistant to such antibiotics. In France, one of the countries which consumes the most antibiotics, many antibiotic-resistant bacteria have been observed. The relationship between cause and effect between these two phenomena appears to be clear, although this has not been rigorously demonstrated. The most important examples include Staphylococci which are among the most resistant bacteria (such as methicillin-resistant Staphylococcus aureus) as well as penicillin-resistant Streptococcus pneumoniae, vancomycin-resistant enterococci, polypharmacoresistant salmonellae and multiresistant tuberculosis bacillae.

Further, imbalances in intestinal flora may arise following an antibiotic treatment (antibiotherapy) intended to combat an infection. Such imbalances themselves cause digestive infections (diarrhea), in particular digestive infections linked to Clostridium difficile (ICD, or CDAD for Clostridium difficile-associated disease). Clostridium difficile is a gram-positive anaerobic bacillum which is the primary cause of nosocomial infectious diarrhea in the adult (approximately 15% to 25% in the case of post-antibiotic diarrhea and more than 95% in cases of pseudomembranous colitis) (Hurley et al. Arch. Intern. Med. 2002; 162: 2177-84), and is naturally partially resistant to the majority of clinically used antibiotics. Oral treatments based on metronidazole or vancomycin are used in the first intention. Patients relapse in 25% of cases, meaning that treatment is lengthy and expensive. Just in the United States, cases of diarrhea arising as a result of antibiotic treatment are estimated to be 12.2 cases per 10000 patients/day between 1987 and 1998, or 7 to 8 cases per 1000 admissions (http://www.cdc.gov/ncidod/dhqp/pdf/infDis/Cdiff_CCJM02_{—}06.pdf). France and Europe are becoming more and more sensitized to this infection by C. difficile since the most virulent strain is spreading into these regions. An epidemic of C. difficile caused by a highly virulent strain occurred in France in March 2006 (source: Institut Francais de Veille Sanitaire; Euro Surveill. 2006 11: E060914.1). People affected by this type of infection died in 0.6% to 1% of cases. These figures are rising and the estimated cost in the United States is of the order of 3669 to 7234 dollars per patient.

Further, currently relatively few biomaterials are protected by antimicrobials. Catheters impregnated with antimicrobials are medical devices for which the most data exists. Essentially, there are two combinations of molecules: chlorhexidine and silver sulfadiazine or minocycline and rifampicin. The in vivo efficacy of the first two is controversial and is reported to be limited time-wise (<10 days). Further, certain cases of anaphylaxia linked to the use of catheters protected by such molecules have been described (Terazawa et al. Anesthesiology, 1998 November; 89(5): 1296-8; Kluger Anaesth Intensive Care, 2003 December; 31(6): 697-8; Stephens et al. Br J Anaesth, 2001 August; 87(2):306-8). A new generation of catheters impregnated with chlorhexidine and silver sulfadiazine not just on the outer face of the catheter but also on the inner face (or lumen) has been shown to reduce colonization (Rupp M E et al, Ann Intern Med. 2005 Oct. 18; 143(8): 570-80). However, the study was not able to demonstrate a significant reduction in cases of septicaemia. Catheters treated with minocycline and rifampicin appear to be more effective, but their use is limited by the risk that abusive use for preventative purposes must cause the appearance of resistant bacteria, resulting in them losing their place in the therapeutic arsenal. This last point is all the more worrying as rifampicin is one of the rare systemic antibiotics which are effective in infections of prostheses.

More than 150 million venous catheters are inserted every year in the United States in hospitals and clinics, i.e. 5 million central venous catheters. In this same country, 80000 stents are used every year. The market for catheters and stents in 2004 was 13.1 billion dollars and is increasing by 12.3% per annum (http://www.marketresearch.com). This advance is faster than that for the drug which is 7% (http://www.interpharma.ch/fr/1926.asp). It is estimated that in 2009 the market will reach approximately 23 billion dollars. It should also be noted that there is a great demand for medical devices that can reduce the risks. The use of stents treated with tacrolimus which reduces the risks of restenosis is increasing faster than conventional catheters. Stents which were treated with molecules which limit infections should thus also find openings. The venous graft market is of the order of 500 million dollars per annum (excluding North America) (http://www.alpha-research.com/coinoanv.html). These data have been summarized in a recent review (Raad et al, Arch Intern Med 2002, 162, 871-879):
rifampicin alone or combined with minocycline is frequently used to treat medical devices. The combination of these two molecules is an effective method since rifampicin is one of those rare antibiotics which are active on slow-growing bacteria, especially on coagulase negative Staphylococci which are the most frequent cause of catheter, stent and venous graft infections. In large multicentre studies, it has been estimated that they reduce the risk of infection by 80% and their use should save a hospital using 850 catheters central venous catheters 500000 $. However, the use of such catheters is slowed by the fear of selecting rifampicin resistant strains in the context of preventative treatment;
silver salts which have a broad antimicrobial spectrum are also used for the treatment of medical devices. That treatment is described in various studies as being ineffective or relatively effective in preventing catheter colonization in place for less than 10 days. They are inoperative in catheters which are placed for a longer period. It would appear that collagen chelates the silver salts and inhibits their activity. Further, they may be responsible for hypersensitivity reactions;
a chlorhexidine-silver sulfadiazine combination: treating catheters with this combination does not appear to be very effective and their long term importance has been hotly debated.

### SUMMARY OF THE INVENTION

The present application concerns a novel class of compounds with an antibiotic activity, more particularly an antibacterial activity with a broad gram-positive spectrum, on bacteria such as Staphylococcus aureus, Staphylococcus epidermidis, Enterococcus faecalis, Bacillus anthracis and Clostridium difficile. The compounds of this class also have a low molecular weight, they are non-toxic in vitro and in vivo and do not cross react with known resistances. Preferably, this class of antibiotic compounds includes margaucine, which may be isolated from a culture supernatant from a bacterial strain.

### BRIEF DESCRIPTION OF THE DRAWINGS

FIG. 1 is the developed formula for margaucine.

### DETAILED DESCRIPTION OF THE INVENTION

The present invention concerns a compound with formula (I) or (II):

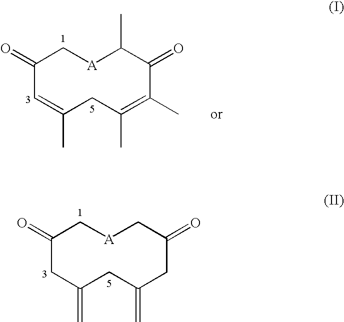

in which A is selected from NR₁, O, S, CR₂R₃ and R—(CH₂)ₙ—R′ in which R and R′, independently of each other, are NH, O, S or CH₂ and R₁, R₂, and R₃ are substituents, and n is equal to 1 or more, in particular equal to 2.

Formulae (I) and (II) as described in this application encompass formulae possibly having substituents:
on the atoms constituting A (such as R₁, R₂ and R₃); and/or
on the carbons in positions 1 and/or 3 and/or 5 (terminal position), such as X₁, X′₁, X₂, X₃ or X′₃.

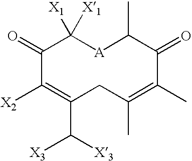

In one particular embodiment of the invention, A is selected from NH, O, S, CH₂ and R—(CH₂)ₙ—R′, in which R and R′, independently of each other, are NH, O, S or CH₂ and n=2.

In one particular embodiment, in the above formulae, the carbon in position 1 is a CH₂, the carbon in position 3 is a CH, and the carbon in the terminal position is a CH₃.

Thus, the present invention concerns a compound with formula (I) or (II):

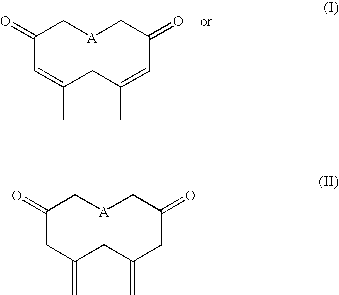

in which A is selected from NH, O, S, CH₂ and R—(CH₂)₂—R′ in which R and R′, independently of each other, are NH, O, S or CH₂.

In a particular embodiment, the invention does not as such envisage the mixture chemically synthesized by the method described by Schank et al (Chemistry of free cyclic vicinal tricarbonyl compounds (“1,2,3-triones”). Part 2. Redox reactions of 1,2,3-triones with ene-1,2-diols (“reductones”), 2-alkoxy-en-1-ols, ene-1,2-diamines, and related species. Helvetica Chimica Acta (2002), 85(5), 1295-1326), i.e. the invention does not pertain to the mixture of compounds with formulae (III) and (IV):

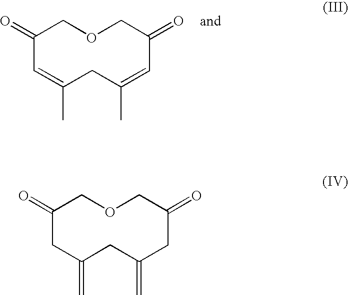

in which the carbon in position 1 is a CH₂, the carbon in position 3 is a CH, the carbon in the terminal position is a CH₃, synthesized under the conditions described by Schank et al. In contrast, the invention pertains to the compounds produced by a bacterium and mixtures thereof, using the method described below by way of example, and concerns the use of said compounds and mixtures regardless of their mode of production (in particular by synthesis or by a bacterium), in particular margaucine, in compositions or applications of said compounds or mixtures described in the present application.

More particularly, the present invention concerns a compound with formula (I) or (II):

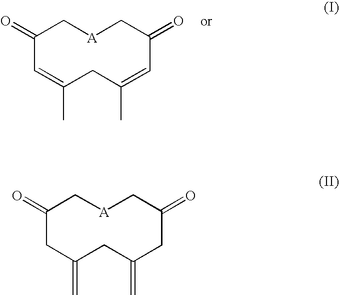

in which A is selected from NR₁, O, S, CR₂R₃ and R—(CH₂)ₙ—R′ in which R and R′, independently of each other, are N, O, S or C and R₁, R₂, and R₃ are substituents, and n is equal to 1 or more, in particular equal to 2. Preferably, A is selected from NH, O, S, CH₂ and R—(CH₂)₂—R′ in which R and R′, independently of each other, are NH, O, S or CH₂.

The particular modes concerning the presence or absence of any substituents on the atoms constituting A and the presence or absence of any substituents on the carbons in positions 1 and/or 3 and/or 5 may be combined independently.

The expression “compound of the invention” encompasses both the compound with formula (I) and the compound with formula (II), i.e. taken individually, regardless of its mode of preparation. In one particular embodiment, the present invention concerns the compound with formula (I), as described in the context of the present application, and in particular the compound with formula (I) produced by a bacterium, and its uses.

The invention also concerns mixtures of these two isomeric compounds, regardless of the proportion of compounds (I) and (II), with the exception of the mixture obtained by chemical synthesis using the method according to Schank et al. The mixtures may comprise forms (I) and (II) even if the antibiotic activity of each of these two forms is not equivalent. Thus, in a particular embodiment, the mixture comprises forms (I) and (II), in which optionally only one form has an antibiotic activity. In a particular embodiment of a mixture of the invention, form (I) has an antibiotic activity.

The present invention also concerns a method for preparing a compound with formula (I) or (II), and more particularly a method for preparing margaucine, comprising (a) culturing a bacterial strain and (b) recovering, in particular purifying, a compound with formula (I) or (II) from the culture supernatant.

In a particular implementation, the bacterial strain used in the context of a method of the invention is the JPL84 or JPL86 strain, and the compound produced is the compound with formula (I).

The term “culturing” means maintaining the bacterial strain, particularly the JPL84 strain, or any derivative strain as defined in the present application, under culture conditions which allow its survival and multiplication. In one particular implementation, culturing is also accompanied by production of the antibiotic with formula (I) or (II) or their mixture or encourages that production. The term “culturing” will be used interchangeably with the term with the same definition, “fermenting”.

Thus, any liquid nutrient medium may be suitable for culturing a bacterial strain, the JPL84 strain or derivative strains provided that that medium contains a source of carbon, nitrogen and inorganic salts. In one implementation, CYE medium (casitone yeast extract) is preferred, which is an agar medium based on yeast extract; its composition is, for example: peptone at 10 g/l, yeast extract at 1 g/l and CaCl₂, at 1 g/l.

Other culture conditions which are known to the skilled person may also be modified, such as the temperature, the presence or absence of stirring of the culture, and the presence or absence of aeration of the culture. In one particular implementation, the bacterial strain, in particular the JPL84 strain, or derivative strains, is cultivated at a temperature in the range 0° C. to 45° C., preferably in the range 20° C. to 37° C., and more preferably 28° C., with stirring and aeration (aerobic culture).

The desired culture period, under the above conditions, depends on the time at which the production of the desired antibiotic is optimized or maximized, and this culture period is calculated with respect to the time at which culturing is begun. Thus, the culture period (period between starting and stopping that culture) is approximately 15 to 24 hours, preferably in the range 18 to 20 hours.

The term “purification” means any technique which can isolate the desired antibiotic from the other constituents of the culture supernatant. By way of non-limiting example, solvent extraction, precipitation, reverse phase chromatography (HPLC), ion exchange chromatography etc, or a combination of two or more of these techniques, may be cited.

The method of the invention may also comprise an optional step between culturing step (a) and purification step (b), consisting of separating cells and other cellular debris from the culture supernatant, for example by centrifuging.

Optionally, the purified antibiotic may then be converted into salts (salt forms) including pharmaceutically acceptable non-toxic salts, by a standard reaction with organic or inorganic acids. These salts will be obtained particularly when A equals N.

Further, the purified antibiotic or salts thereof may be obtained in the anhydrous form by lyophilization.

The present invention also envisages a compound with formula (I) or (II) or a mixture thereof as described above, obtained by a method involving culturing bacteria as described in this application.

A mixture of isomers with formulae (I) and (II) as described above may also be obtained by chemical synthesis such as, for example, that disclosed in the publication by Schank et al. (Chemistry of free cyclic vicinal tricarbonyl compounds (“1,2,3-triones”). Part 2. Redox reactions of 1,2,3-triones with ene-1,2-diols (“reductones”), 2-alkoxy-en-1-ols, ene-1,2-diamines, and related species. Helvetica Chimica Acta (2002), 85(5), 1295-1326). That synthesis results in the production of a mixture from which one of the compounds with formula (I) or (II) may be isolated.

When the compound of the invention is obtained by a bacterial culture method as described above, it conserves a trace of its bacterial production and may be distinguished from the same compound produced by chemical synthesis because of the observed difference in their respective C₁₂/C₁₃ ratios. Thus, the chemically produced compound with formula (I) or the compound with formula (II) has a C₁₂/C₁₃ ratio which is different from the compound with formula (I) or compound with formula (II) obtained by a bacterial method, and in particular different from the compound with formula (I) or compound with formula (II) obtained from bacteria cited in the present application. In a particular implementation, the chemically produced margaucine has a C₁₂/C₁₃ ratio which is different from the margaucine obtained by a bacterial method, and in particular is different from the margaucine obtained from the bacteria cited in the present application.

A composition comprising at least one compound with formula (I) or (II) as described above also falls within the scope of the invention, with the exception of the mixture of compounds with formulae (I) and (II) when it is obtained by chemical synthesis using the method published by Schank et al.

In a particular implementation, a composition in accordance with the invention comprises at least one compound with formula (I) or (II) obtained by a production method in a bacterium described above, particularly a composition comprising at least the compound with formula (I) obtained from the strain JPL84.

Further, the invention concerns a composition which comprises at least one transporter and/or a vehicle, and a compound with formula (I) or (II):

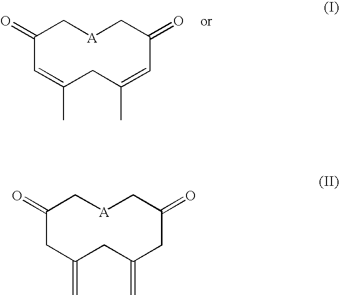

in which A is selected from NR₁, O, S, CR₂R₃ and R—(CH₂)ₙ—R′ in which R and R′, independently of each other, are NH, O, S or CH₂ and R₁, R₂, and R₃ are substituents, and n is equal to 1 or more, in particular equal to 2. In a particular embodiment, the composition comprises at least one vehicle and at least the compound with formula (I), in particular margaucine (compound with formula III). In this composition, the compound is obtained by chemical synthesis or by a method comprising culture of bacteria.

The term “vehicle” means any substance which can allow the antibiotic to be formulated into a composition. In one preferred embodiment, the vehicle is a substance or a combination of physiologically acceptable substance(s), i.e. appropriate for use of the composition in contact with a living being (for example a non-human mammal, and preferably a human being) and is thus preferably non-toxic. Examples of such physiologically acceptable vehicles are water, saline solution, solvents which are miscible with water, sugars, binders, excipients, pigments, vegetable or mineral oils, water-soluble polymers, surfactants, thickening or gelling agents, cosmetic agents, preservatives, alkalinizing or acidifying agents, etc. Particular compositions of the invention are pharmaceutical compositions, i.e. they are formulated for administration or application to a living being, for therapeutic or prophylactic purposes. These pharmaceutical compositions contain pharmaceutically acceptable vehicles for local or systemic administration, particularly by injection, for application in contact with a living being by ingestion, or use in the solid form, gel form, liquid form or aerosol form. Alternatively, the compositions of the invention are applied to materials which come into contact with a living being as defined above. In one particular implementation, the composition comprises a pharmaceutically acceptable vehicle and margaucine.

The invention also concerns a composition which comprises at least one compound with formula (I) or (II) as defined in the present application, if appropriate associated with a transporter and/or a vehicle as defined in the present application, and which also comprises at least one second molecule, different, active against micro-organisms. The expression “active molecule” means a chemical or biological compound, in particular a compound with a low molecular weight, for example a peptide, which has the capacity to destroy, neutralize the activity or prevent the proliferation of micro-organisms. This active molecule may, for example, be an antibiotic or a bactericide. The active molecule is preferably selected from a compound which is active against gram-negative bacteria and/or a fungicidal compound and/or the compounds listed in Table 3 below or the following compounds: silver salts, minocycline, chlorhexidine, sulfadiazine, rifampicin, etc.

In a particular embodiment, when the compound of the invention, alone or as a mixture or when a composition of the invention is used to treat digestive infections linked to C. difficile (ICD), it is not necessary to administer it in combination with another antibiotic, such as those cited in Table 6, for example. In the treatment of ICD, the compound of the invention has a bactericidal activity against C. difficile without in any way perturbing the intestinal flora; co-administration of other antibiotics could cancel out the surprising effect of the compound of the invention as regards preserving the intestinal flora.

In another embodiment, a composition of the invention comprises at least two compounds with formula (I) or (II) as defined above, but which differ from each other in their structure, namely their atomic composition or the position of the various substituents. As an example, the composition comprises at least two compounds with formula (I) which differ in the nature of the element A, in the substituents located on the element A and/or in the substituents in positions 1, 3 and/or 5. Another composition of the invention comprises at least two compounds with formula (II) which differ by the nature of the element A, in the substituents located on the element A and/or in the substituents in positions 1, 3 and/or 5.

The invention also envisages a composition which comprises at least one compound with formula (I) and a compound with formula (II), in which A is identical in the two compounds and is selected from NH, S, CH₂ and R—(CH₂)₂—R′ in which R and R′, independently of each other, are NH, O, S or CH₂, i.e. comprises two isomers of the same compound as regards the position of the double bonds.

In a particular embodiment, the compositions of the invention also comprise a vehicle or a transporter.

The present invention also pertains to the use of a compound with formula (I) or (II) taken individually or as a mixture or a composition as defined above, as a drug, and more particularly as an antibiotic (which destroys or prevents the proliferation of micro-organisms) and/or bactericide (which destroys or prevents the proliferation of bacteria).

In a particular embodiment, the compound with formula (I) or (II) taken individually or as a mixture or the composition of the invention has a bactericidal activity against gram-positive bacteria (gram staining) or bacteria having a thin wall composed of several layers including a lipid, i.e. the compound destroys the bacteria or slows their growth.

In a particular embodiment, the compound with formula (I) or (II) taken individually or as a mixture or the composition of the invention is used to treat infections caused by bacteria of the genus Clostridium. More particularly, the compound with formula (I) or (II) taken individually or as a mixture or the composition of the invention is used to treat infections caused by Clostridium difficile, and preferably digestive infections linked to Clostridium difficile (ICD) such as simple diarrhea, post-antibiotic diarrhea, or pseudomembranous colitis (PMC). Preferably, the use as a drug concerns the compound with formula (I) or that with formula (III).

Thus, the invention concerns a compound, a mixture or a composition of the invention, more particularly margaucine or a composition comprising it, for use as a drug, preferably as an antibiotic or a bactericide.

In its therapeutic uses or therapeutic methods (which include administration to a living being of at least one compound with formula (I) or (II) taken individually or as a mixture or of a composition of the invention), the compound(s), the mixture or the composition comprising it (them), may be in the solid form (wafer, powder, gelule, pill, suppository, rapid release tablet, gastroresistant tablet, slow release tablet), gelatinous form (gel, pomade, cream, ovule), liquid form (syrup, injectable solution, eye lotion) or aerosol form (spray, vapor, gas). Thus, depending on its galenical form, the compound, the mixture or the composition of the invention may be administered orally, transmucous-buccally, nasally, ophthalmically, otologically (into the ear), vaginally, rectally, parenterally (intravenously, intramuscularly, or subcutaneously), transcutaneously (or transdermally or percutaneously) or cutaneously. One administrative or galenical form which can encourage the bactericidal activity of the compound with formula (I) or (II) taken individually or as a mixture or a composition of the invention in the intestine constitutes a particular implementation of the invention.

The administered doses are those conventionally used for other antibiotics or combinations of antibiotics. By way of indication, doses of from 30 mg/kg/day to 100 mg/kg/day, depending on the age or weight of the patient, the severity or stage of the infection, the number of daily administrations or the mode of administration, may be administered; principally in the adult, doses of between 1 g/day and 10 g/day are normal.

The present invention also concerns the use of a compound with formula (I) or (II) taken individually or as a mixture, for the manufacture of a composition for use in combating bacterial infections, in the treatment or prophylactic treatment of bacterial infections, and more particularly of diseases which are resistant to antibiotics administered until now.

The expression “resistant diseases” means any infection the origin of which is entirely or partially bacterial. More particularly, the expression “resistant diseases” as used in the context of the present application encompasses infections wherein at least one responsible agent is sensitive to an antibiotic compound of the invention, whether it is used alone or in combination with another compound of the invention or another active molecule. Examples which may be cited are multi-resistant diseases (resistant to several classes of antibiotics) or all infections caused by gram-positive bacteria.

The term “treatment” also encompasses both the curative effect (destruction of the micro-organism) obtained with at least one compound of the invention or a composition of the invention, and the improvement in symptoms observed in the patient (and linked to the presence of a micro-organism or micro-organisms) or an improvement in the state of the patient. Thus, the term “treatment” is applicable to the principal or secondary point of infection, like the symptoms resulting from infection.

The compounds, mixtures and compositions of the invention are particularly used in the treatment of nosocomial infections.

The term “prophylaxis” means any use of at least one compound, mixture or composition of the invention, for preventative purposes, i.e. aiming to prevent the appearance of symptoms after contamination or suspicion of contamination, or to prevent infection by a micro-organism or its consequences, particularly in the case of an infection due to a bacterium, more particularly a gram-positive bacterium, and more particularly an infection due to a bacterium of the genus Clostridium such as digestive infections linked to Clostridium difficile.

The compounds, mixtures or compositions of the invention are used in therapy and prophylaxia using the galenical forms and modes of administration indicated above. In one particular implementation, the compound used is margaucine or any composition as defined in the present application comprising at least margaucine.

The invention also pertains to the use of compounds, mixtures or compositions of the invention in cosmetic, food or veterinary applications (domestic animals, poultry, swine, sheep, cattle or pets).

The compound with formula (I) or (II) of the invention, taken individually or as a mixture or a composition of the invention is particularly effective against bacteria of the genus Clostridium, especially the toxinogenic forms, examples of which are Clostridium difficile, Clostridium perfringens, Clostridium botulinum, Clostridium tetani, Clostridium novyi, Clostridium histolyticum, Clostridium butyricum, Clostridium septicum, Clostridium sordellii, Clostridium ramosum, Clostridium bifermentans, Clostridium paraperfringens, Clostridium cadaveric, Clostridium clostridiiforme, Clostridium innocuum, Clostridium limosum, Clostridium paraputrificum, Clostridium sporogenes, Clostridium subterminale, Clostridium tertium, Clostridium baratii, Clostridium argentinense, Clostridium chauvoei, Clostridium haemolyticum or Clostridium spp. With respect to the Clostridium difficile bacteria, the invention envisages the treatment of infections caused by the toxinogenic forms of C. difficile, such as the epidemic strain of PCR-ribotype027. Because of its particularly effective action on the bacteria of the genus Clostridium such as Clostridium difficile (MIC of the order of 0.05 μg/ml) on the one hand, and its greater activity as regards bacteria of the genus Clostridium with respect to other bacteria of the intestinal flora (30 times more active) on the other hand, the compound with formula (I) or (II) of the invention taken individually or as a mixture or a composition of the invention, and in particular margaucine (III) is particularly advantageous for use in restoring the equilibrium between various intestinal bacterial populations (microbial flora). Thus, the present invention also envisages the use of a compound with formula (I) or (II) of the invention taken individually or as a mixture or a composition of the invention, in particular margaucine (III), to treat infections due to bacteria of the genus Clostridium, more particularly infections due to C. difficile, such as ICDs. In one particular embodiment, the compound with formula (I) or (II) of the invention taken individually or as a mixture or a composition of the invention is used to treat ICDs consecutive to antibiotic therapy. In a preferred embodiment, this compound is margaucine or the composition comprises margaucine (compound with formula (III)).

Therapeutic and prophylactic methods which are particularly envisaged in the present invention are where patients present with an infection caused by C. difficile, and in particular (a) patients with an ICD; (b) patients with a severe ICD, (c) patients with a relapse of ICD, the relapse being characterized by a new episode of ICD appearing in the 8 weeks following the onset of the preceding episode, whether the strain of C. difficile is the same or different from that of the preceding episode. The therapeutic and prophylactic methods are applicable to both nosocomial ICDs (patient hospitalized or not hospitalized leaving a health establishment less than 4 weeks previously) and to communal ICDs. In particular, these methods are thus aimed at patients from health establishments, retirement homes or medico-social establishments, and all patients presenting with risk factors as regards ICD, namely patients having antibiotic therapy (more than 90% of cases), persons aged over 65 years, persons with a severe subjacent disease, patients who have undergone nasogastric intubation, patients who have received anti-ulcer drugs and long-term hospitalized patients.

The invention also concerns the use of a compound with formula (I) or (II) taken individually or as a mixture or a composition as defined above, for the impregnation of devices, including medical devices (or biomaterial). The term “impregnation” means the application of at least one compound, mixture or a composition of the invention, and possibly its penetration (deep or superficial) onto the device. Thus, the invention also envisages devices impregnated with compound (alone, as a mixture or as a composition) of the invention. In a particular embodiment, the devices are biomedical devices, i.e. devices for medical use, non-limiting examples of which are catheters, dressings, bone cements, cerebral shunts, cardiac valves, etc. Preferred devices for biomedical use are devices produced from polymeric material such as polyethylene, polypropylene, polyvinyl chloride, polycarbonate, polyurethane, acrylate or methacrylate or polyamide or materials obtained by weaving. In one particular embodiment, these devices are biocompatible.

In another embodiment, the impregnated device is a fabric for domestic or industrial use, such as a bandage.

The invention also pertains to bacterial strains which have the capacity to produce at least one compound with formula (I) or (II) of the invention. In a particular embodiment, the strains produce the compounds of the invention under fermentation conditions which are disclosed in section A below (method and apparatus). These strains may produce a compound with formula (I) or (II), taken individually or as a mixture, in particular margaucine. As an example, such a strain is the JPL84 strain deposited under the Treaty of Budapest at the C.N.C.M. (Collection Nationale de Cultures de Microorganismes; Institut Pasteur; 25, rue du Docteur Roux; 75724 Paris Cedex 15; France) on 15 Sep. 2006 with accession number CNCM I-3669. In one embodiment, which may or may not be in combination with the preceding embodiments, the bacterial strains are Bacillus strains.

The invention also pertains to the strains derived from the strains described above, in particular to any strain derived from JPL84 strain or JPL86, characterized in that they conserve the capacity to produce a compound with formula (I) or (II) or a mixture, and more particularly in that they conserve the capacity to produce margaucine. The term “derivative” means any strain which is not found in the natural state and obtained from natural strains by modification of its genotype, in particular a strain obtained by recombination (recombinant strain) provided that it continues to produce a compound with formula (I) or (II) of the invention. In a particular embodiment, the recombinant strain produces a larger quantity of compounds of the invention than the natural strain. The modification of the genetic inheritance may, inter alia, consist of modification of enzymes and other proteins involved in the production and secretion of the compound of the invention.

The invention also concerns a method for modulation of the bacterial profile of a biological sample or of a surface, comprising bringing said biological sample or said surface into contact with at least one compound in accordance with formula (I) or (II) taken individually or as a mixture or a composition of the invention, under conditions which can modify the bacterial profile; the method may also comprise a second step for establishing the modulation.

Within the context of the invention, the expression “modulate the bacterial profile” means modifying the ratio existing between the various bacterial strains present in the biological sample or on the surface, before and after contact with the compound or composition of the invention.

The method of the invention is appropriate for treating the surfaces of various objects, such as the surfaces of objects from medical services (for example in a hospital medium).

Thus, when the biological sample or the surface contains only bacteria which are sensitive to the compound of the invention, or when the strains contained in a biological sample or on the surface are sensitive to a composition of the invention, the expression “modulate the bacterial profile” encompasses neutralizing the bacterial activity, in particular destruction of all the bacteria, i.e. eliminating any bacterial profile.

In contrast, when the sample or the surface comprises strains which are resistant or less sensitive to the compound or composition of the invention and strains which are sensitive to that same compound, the expression “modulate the bacterial profile” means entraining a modification of the ratio between the strains resistant to the compound of the invention and the more sensitive strains. As an example, addition of a compound of the invention causes a reduction in gram-positive strains, which modifies the gram-positive/gram-negative strain ratio.

### EXAMPLES

### A. Methods and Apparatus

### JPL84 Strain

Margaucine, which is the compound with formula (III):

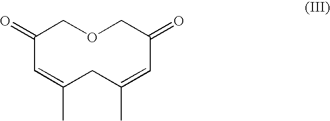

in which the carbon in position 1 is a CH₂, the carbon in position 3 is a CH and the carbon in the terminal position is a CH₃, is produced by the JPL84 strain, a strain isolated in 2005 from dirt in the Agout region, in France. This strain was deposited on 15 Sep. 2006 at the C. N.C. M. (Collection Nationale de Cultures de Microorganismes; Institut Pasteur, Paris, France), with accession number CNCM I-3669. Sequencing of the gene coding for the RNA 16S has been carried out; the JPL84 strain could be a strain of Bacillus sp. The strain was maintained at 4° C. on an inclined CYE medium agar gel (CYE agar) containing 10 g of casitone, 1 g of yeast extract, 1 g of CaCl₂ and 14 g of agarose gel, in 1 liter of water.

### Fermentation or Culturing

Fermentation of this strain was carried out in a CYE medium containing 10 g/l of peptone, 1 g/l of yeast extract and 1 g/l of CaCl₂. In order to produce margaucine, the fermentation was carried out in 10 l of CYE medium as defined above, at 28° C. with stirring and aeration. Margaucine started to be produced after fermentation for 14 h and reached its peak between 18 and 20 h. Production of the antibiotic was monitored and quantified by a diffusion test on agar gel against Staphylococcus aureus (CIP 76.25) and by analytical high pressure liquid chromatography (HPLC).

### Purification

The molecule was purified by reverse phase chromatography: acetonitrile (10% v/v) as well as fixing beads (Amberlite XAD-16) were added to the culture. It was kept at 4° C., with stirring, for 12 h. The XAD-16 beads were separated from the culture, washed with water and a water/methanol mixture (50/50) then eluted with methanol (100%). This eluate was concentrated by vacuum evaporation. Finally, the margaucine was purified by reverse phase HPLC on a C18 preparative column using a H₂O/0.1% TFA linear gradient and acetonitrile/0.1% TFA from 20% to 80% of acetonitrile/0.1% TFA in 30 min with a flow of 10 ml/min. After lyophilization, 30 mg of margaucine was obtained from 10 L of culture.

### Determination of Structure

The molecular formula was determined by a combination of spectroscopic techniques: one- and two-dimensional NMR and mass spectrometry.

The ¹³C, DEPT, ¹H, COSY, HMQC and HMBC analyses which were carried out in CD3OD medium with a Bruker Advance DPX-300 provided with a direct 5 mm QNP probe operating at 300 MHz for ¹H and at 75 MHz for ¹³C are shown in Table 1.

### Biological Properties

a. Determination of Minimum Inhibiting Concentrations (MIC) and Test on Multiresistant Bacteria

The minimum inhibiting concentrations (MIC) were evaluated for the reference strains by microdilution in Mueller Hinton Broth (MHB) medium in accordance with CLSUNCCLS standards (National Committee for Clinical Laboratory Standards (2003). Methods for dilution antimicrobial susceptibility tests for bacteria that grow aerobically—sixth edition: Approved standard M07-A6. NCCLS, Villanova, Pa., USA.

The MIC was determined as the lowest concentration of margaucine which inhibits all visible culture of a bacterial strain, after 18 hours of culture at 37° C.

The antimicrobial activity of the margaucine is shown in Tables 2 and 3.

b. Evaluation of In Vitro Toxicity of Margaucine

The in vitro toxicity of margaucine was determined on a breast cancer cell line (MCF7); briefly, a 96-well culture dist was cultured with MCF7 cells (ATCC HTB-22™, approximately 10000 cells/well) in a RPMI medium containing 10% foetal calf serum (t=0). At t+1 (in days, with respect to start of culture), the test molecule was added. At t+2, the cytotoxicity was measured by incorporating MTT (3-(4,5-dimethylthiazol-2-yl)-2,5-diphenyl tetrazolium bromide) in accordance with Heeg K et al (J Immunol Methods 1985, March 18; 77(2): 237-46). The cytotoxicity results are shown in Table 4.

c. Evaluation of In Vivo Toxicity of Margaucine

Three (3) female 25 g OF1 mice (Charles River) received, by intraperitoneal injection, 200 μl of a solution of margaucine in an amount of 12.5 mg/ml in 10% methanol and 90% H₂O (i.e. a dose of 100 mg/kg). Survival of the mice was carried out at T24, T48, T72 and T96 (time in hours).

d. Determination of In Vivo Efficacy of Margaucine on Bacterial Infection

The molecule was tested on a model of septicaemia with Staphylococcus aureus, after infection of OF1 mice by intraperitoneal injection of bacteria.

For a total of 11 female 25 g OF1 mice (Charles River), at time T0, 3 mice received an intraperitoneal injection of 200 μl of LB+5% of mucine (monitoring breeding conditions) and 8 mice received one intraperitoneal injection of 200 μl of a bacterial suspension of Staphylococcus aureus Smith (10⁸ CFU/ml) in LB medium+5% mucine (infected mice).

The 8 infected mice were divided into 2 batches: at t+3 hours, 5 mice received a solution of margaucine in an amount of 12.5 mg/ml in 10% methanol and 90% H₂O (i.e. a dose of 100 mg/kg) while the other 3 mice did not receive margaucine (infection monitoring).

Survival of the mice was checked at 24 and 48 hours.

### B. Results

### Structure

The structural analyses demonstrated that the empirical formula of margaucine is C₁₂H₁₈O₃.

The ESI-MS analysis provided a molecular mass of 210 Da for the margaucine.

¹³C NMR exhibited 5 signals (40.4, 71.6, 95.6, 105.0 and 170.2) and ¹H NMR showed two groups of signals (Table 1):
a first group with 3 ¹H signals (at 2.66, 3.63 and 5.81 ppm) corresponding to protons bound to carbons with a visible resonance on the ¹³C spectrum, all of the signals being singlets; and
a second group with 2 ¹H signals with a chemical shift which was almost identical to the first group, bound to carbons with a resonance which was not visible on the ¹³C spectrum. They corresponded to an alcoholic form of margaucine.

An unprotected methylene group at 3.63 ppm could be attributed to the presence of an oxygen atom and a carbonyl group in the a position.

The COSY spectrum did not show any correlation since the protons are in the position a to a quaternary carbon or a heteroatom.

The presence of two quaternary carbons (170.2 and 105 ppm), a methine (95.6 ppm), a methylene (71.6 ppm) and two equivalent methyl groups (40.4 ppm) was deduced from the DEPT spectrum (Table 1).

The attribution of the protons bonded to these carbons via a ¹³C-¹H bond was obtained using the HMQC spectrum. The structure of the margaucine was confirmed by the HMBC spectrum and its long distance scalar interactions with the linked peaks C1/H3, C2/H3 and C2/H5 (Table 1).

TABLE 1
¹H and ¹³C NMR, DEPT, HMQC
and HMBC data for margaucine in CD₃OD.

| Carbon no | δ ¹³C/ppm | DEPT/HMQC | δ 1H/ppm | I* | HMBC |
|---|---|---|---|---|---|
| 1 | 170.2 | C | — | — | C1/H3 |
| 2 | 105.0 | C | — | — | C2/H3 |
| 3 | 95.6 | CH | 5.81 | S | — |
| | | | 5.79 | | |
| 4 | 71.6 | CH2 | 3.63 | S | C4/H4 |
| | | | 3.62 | | |
| 5 | 40.4 | CH3 | 2.66 | S | C5/H5 |
| 6 | 33 | CH3 | 2.63 | S | |

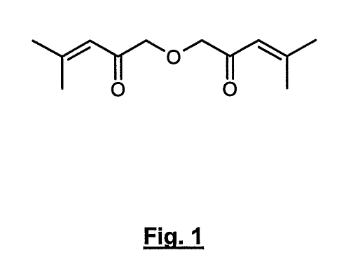

The combination of NMR data and the mass information suggests that the molecule is symmetrical. The MS/MS experiments carried out on the molecular ion with m/z=211 produced a set of fragments which were in complete agreement with the proposed structure of FIG. 1 (ions obtained: 193, 175, 169, 165, 153, 151, 147, 141, 133, 129, 123, 119, 111, 109, 105, 95, 93, 91, 85, 83, 81, 79, 71, 69, 67, 65, 57, 55, 43). The UV absorption spectrum showed two maxima—λmax: 269 and 330.

The structure of margaucine is shown in FIG. 1 and is termed 1,1′-oxybis[4-methylpent-3-en-2-one].

### Minimum Inhibiting Concentrations (MIC)

The MIC of margaucine for various gram-positive or gram-negative bacteria as well as for yeasts were determined and are shown in Table 2.

Margaucine is active only against gram-positive bacteria but does not have any antibiotic activity towards gram-negative bacteria nor antifungal activity towards Candida albicans (yeast). Very importantly, the present experiment also demonstrates that the E. coli to IC strain (gram-negative bacterium) is sensitive to margaucine. This toIC strain is deficient as regards an efflux pump system, which means that it may be hypothesized that the cause of the inactivity of margaucine as regards gram-negative bacteria is linked to the problem of penetration of the bacterial wall (composed of several layers including a lipid layer in gram-negative bacteria, while it is composed of a single thick layer of mureine or peptidoglycan in gram-positive bacteria). Further, Table 2 shows that the molecule is effective against gram+ bacteria at concentrations as low as 3.125 μg/ml, or even 0.05 μg/ml for Clostridium difficile.

TABLE 2
Minimum inhibiting concentration (MIC) of margaucine with regard
to various gram-positive (+) or gram-negative (−) strains and
of Candida albicans (n.a: non applicable; CIP: collection Institut
Pasteur; ATCC: American type culture collection).

| Organism | Gram | MIC (μg/ml) |
|---|---|---|
| Bacteria | | 0.05 |
| Clostridum difficile DSM1296 | + | 3.125 |
| Staphylococcus aureus CIP 76.25 | + | 3.125 |
| Staphylococcus epidermidis | + | 3.125 |
| Staphylococcus aureus H1 | + | 3.125 |
| Staphylococcus aureus H3 | + | 1.563 |
| Staphylococcus aureus H4 | + | 3.125 |
| Staphylococcus aureus H7 | + | 3.125 |
| Staphylococcus aureus H9 | + | 3.125 |
| Staphylococcus aureus H16 | + | 3.125 |
| Staphylococcus aureus H18 | + | 3.125 |
| Enterococcus faecalis H | + | 3.125 |
| Bacillus anthracis | + | 3.125 |
| Bacillus subtilis ATCC 27370 | + | 6.25 |
| Escherichia coli CIP 76.24 | − | >100 |
| Escherichia coli ToIC | − | 3.125 |
| Salmonella typhi | − | >100 |
| Serratia marcescens | − | >100 |
| Pseudomonas aeruginosa CIP 76.110 | − | >100 |
| Yeast | | |
| Candida albicans | n.a. | >100 |

Finally, it was shown that margaucine was also active against multiresistant gram-positive strains such as various strains of Staphylococcus aureus (H1, H3, H4, H7, H9, H16 and H18) (Table 3).

TABLE 3
Susceptibility of various strains of Staphylococcus aureus
to various antibiotics including margaucine.

| | Staphylococcus aureus | | | | | | |
|---|---|---|---|---|---|---|---|
| | H1 | H3 | H4 | H7 | H9 | H16 | H18 |
| Penicillin G | R | R | R | R | R | R | R |
| Amoxicillin | R | R | R | R | R | R | R |
| Piperacillin | R | R | R | R | R | R | R |
| Oxacillin | S | S | R | S | S | R | R |
| Cefazoline | S | S | R | S | S | R | R |
| Tobramycin | S | S | R | S | S | R | R |
| Amikacin | S | S | R | S | S | R | R |
| Gentamicin | S | S | S | S | S | R | S |
| Tetracycline | S | S | S | — | — | R | — |
| Doxycyclin | S | S | S | S | S | L | S |
| Erythromycin | S | R | S | R | R | R | R |
| Clindamycin | S | R | S | R | S | R | R |
| Pristinamycin | S | S | S | S | S | L | S |
| Co-trimoxazole | S | S | S | S | S | S | S |
| Pefloxacine | — | — | R | R | S | — | R |
| Ofloxacin | S | R | R | R | S | R | R |
| Norfloxacin | S | R | R | R | S | R | R |
| Ciprofloxacin | — | — | R | R | S | — | R |
| Rifampicin | S | S | S | S | S | R | S |
| Fusidic acid | S | S | S | S | R | S | S |
| Fosfomycin | S | S | S | S | S | R | R |
| Vancomycin | S | S | S | S | S | S | R |
| Teicoplanin | S | S | S | S | S | L | R |
| Margaucine* | 3.125 | 3.125 | 3.125 | 1.563 | 3.125 | 3.125 | 3.125 |

(R: strain resistant to antibiotic concerned; S: strain sensitive to antibiotic concerned; L strain with intermediate resistance to antibiotic concerned; —: not tested;
*MIC determined using method described above.

No cross resistance was observed with commercial antibiotics; further, no spontaneous resistant strain was isolated, even after random mutagenesis.

### Toxicity In Vitro and In Vivo

Before envisaging the use of margaucine in vivo to combat gram-positive bacteria, toxicity tests were carried out in vitro (cell culture) and in vivo (animal).

Firstly, and as can be seen in Table 4, no cytotoxicity was measured on a cell culture of MCF7 cells treated with margaucine in a concentration from 1 to 100 mg/ml. In fact, the number of cells in a culture treated by margaucine was identical to that of a control culture (received no antibiotic).

TABLE 4
Cytotoxicity of margaucine on a culture of MCF7 cells

| | OD |
|---|---|
| Control (no margaucine) | 1.4 ± 0.1 |
| Margaucine 1 mg/ml | 1.3 ± 0.2 |
| Margaucine 10 mg/ml | 1.2 ± 0.2 |
| Margaucine 100 mg/ml | 1.3 ± 0.1 |

Further, injection of margaucine in a dose of 100 mg/kg of animal did not give rise to any toxicity in 3 mice which were alive after 96 hours. Thus, these results confirm that margaucine is harmless in vivo.

As a result, margaucine is not toxic for eukaryotic cells both in vitro and in vivo at concentrations as high as 100 mg/kg.

### Efficacy In Vivo

Finally, the efficacy of margaucine in vivo was evaluated on a murine model of septicaemia with Staphylococcus aureus. The results are summarized in Table 5.

TABLE 5
Survival of mice at 24 and 48 hours post-infection (−: no; +: yes).

| Infected | Mice receiving | Number | Survival | |
|---|---|---|---|---|
| mice | margaucine | of mice | At 24 h | At 48 h |
| − | − | 3 | 3 live mice | 3 live mice |
| + | − | 3 | 3 dead mice | / |
| + | + | 5 | 3 live mice | 3 live mice |
| | | | 2 dead | 2 dead |

These results demonstrate that infection with Staphylococcus aureus was fatal in 100% of the mice which had not received antibiotic. In contrast, 3 of the 5 mice which had received 100 mg/kg of margaucine survived the infection.

As a consequence, these results confirm on the one hand the in vitro MIC tests and on the other hand that margaucine has an antibiotic activity in vivo against gram-positive bacteria such as Staphylococcus.

Comparison of MIC of Several Molecules on C. difficile

Table 6 below summarizes the minimum inhibiting concentration (in μg/ml) determined for C. difficile of other molecules which are in current use or are in clinical trials (phase III).

TABLE 6
Comparison of activity of various molecules on C. difficile

| Molecule | MIC | Remarks |
|---|---|---|
| Margaucine | 0.05 | This application |
| Metronidazole | 1 | Bishara et al, Diagn Microbiol Infect |
| | | Dis 2006 February; 54(2): 141-4 |
| Nitazoxanide | 0.1 | Catherine et al, Antimicrob |
| | | Agents Chemother 2000 |
| | | September; 44(9): 2254-2258 |
| Vancomycin | 1 | In current use |
| Rifaximin | 0.08-0.2 | Also known as Xifaxan; phase III |
| Lipiarmycin | 0.06-2 | Also known as OPT-80; phase III |
| | | Finegold et al, Antimicrob |
| | | Agents Chemother 2004 December; |
| | | 48(12): 4898-902 |

In Vivo Efficacy of Margaucine on Infections with C. difficile

The molecule was tested on a model of infection by C. difficile after subcutaneous injection of clindamycin (10 mg/kg of body mass) to induce colitis, in golden Syrian hamsters weighing 60 g to 80 g (batches of 20 animals). Simultaneously with the injection of clindamycin or 24 hours after this injection, the hamsters were inoculated orally with a virulent clinical strain of C. difficile (10⁵ strains per animal). Alternatively, the virulent strain of C. difficile was the strain ATCC 43255.

One hour after inoculation with C. difficile, the hamsters were treated with margaucine by gavage (10 to 200 mg/kg of body mass). This treatment was repeated for 3 days, once a day with the same quantity of margaucine.

Controls were used under the same conditions: batches of 20 animals not infected with C. difficile and/or not treated with margaucine.

The experiments are currently under way; the animals are being observed for a period of 30 days and moribund animals are being counted then sacrificed.

### CONCLUSION

A novel compound, margaucine, has been identified having antibiotic activity and produced by a bacterial strain. This compound is characterized structurally and is of low molecular weight. Further, margaucine has a broad gram-positive spectrum, is non-toxic, effective on animal infection models and does not cross with any known resistance. Other compounds with an analogous structure, and having an antibiotic activity, are also proposed in the present application, to illustrate the importance of this class of compounds for antibiotic applications.

Because of its particularly effective action on bacteria of the genus Clostridium such as Clostridium difficile, at low concentrations (MIC of the order of 0.05 μg/ml), the compounds or compositions of the invention, and more particularly margaucine, represent a high therapeutic potential against digestive infections linked to C. difficile and more particularly intestinal infections consecutive to antibiotic therapy and due to C. difficile. Further and in contrast to commercial antibiotics, the compound or composition of the invention is 30 times more active on C. difficile bacteria than on the other bacteria of the intestinal flora. These results make the compounds or compositions of the invention an antibiotic of choice in the treatment of infections by C. difficile compared with commercial antibiotics.

By way of comparison, molecules which are currently used to treat infections with C. difficile are as follows:
(a) vancomycin, used to treat the most severe cases (MIC of the order of 1 μg/ml on C. difficile); however, vancomycin is already used to treat other most severe bacterial infections, in particular due to Staphylococcus, and has led to the appearance of many resistant strains; it use is restricted as much as possible by the medical community in order to avoid the spread of resistance and the emergence of new resistant strains; and
(b) metronidazole used in the first intention but which is inactive for 25% of patients and for which frequent relapses have been recorded (25% of cases); furthermore, although metronidazole is cheaper than vancomycin, it has more side effects.

Other molecules which are currently undergoing clinical trials may also be compared with the compound of the invention:
(a) nitazoxanide (phase III, Romark Laboratories LC), a molecule the action of which resembles metronidazole; however, this molecule is not recommended for pregnant or lactating women;
(b) rifaximin (Xifaxan) (phase III, Salix Pharmaceuticals), a molecule derived from rifampicin; its minimum inhibiting concentration (MIC) is of the order of 0.08 to 0.2 μg/ml on C. difficile and this molecule generates many spontaneous resistances; and
(c) lipiarmycin (OPT-80) (phase III, Optimer Pharmaceuticals), the minimum inhibiting concentration (MIC) of which is of the order of 0.06 to 2 μg/ml for C. difficile (Antimicrob Agents Chemother, 2004 December; 48(12); 4898-902).

As a consequence, the compound of the invention or a composition comprising it, and particularly margaucine, constitutes a seductive alternative proposition to therapeutic molecules already on the market or to those currently under clinical trials, in the treatment of digestive infections linked to C. difficile (ICD).

1.-37. (canceled)

38. A compound with formula (I) or (II): 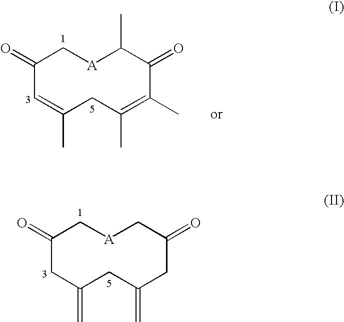
in which A is selected from NR₁, S, CR₂R₃ and R—(CH₂)ₙ—R′ in which R and R′, independently of each other, are NH, O, S or CH₂ and R₁, R₂ and R₃ are substituents, and n is equal to 1 or more, in particular equal to 2.

39. The compound with formula (I) or (II) according to claim 38, in which A is selected from NH, S, CH₂ and R—(CH₂)₂—R′, in which R and R′, independently of each other, are NH, O, S or CH₂.

40. A method for preparing a compound with formula (I) or (II): 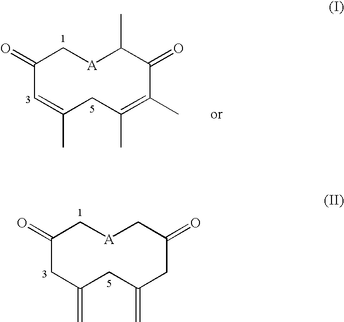
in which A is selected from NH, S, O, CH₂ and R—(CH₂)₂—R′ in which R and R′, independently of each other, are NH, O, S or CH₂, comprising:
a) culturing a bacterial strain; and
b) purifying the compound with formula (I) or (II) from the culture supernatant.

41. The preparation method according to claim 40, in which the bacterial strain is the JPL84 strain deposited at the CNCM with accession number CNCM I-3669.

42. The preparation method according to claim 40, in which the compound produced has the following formula:

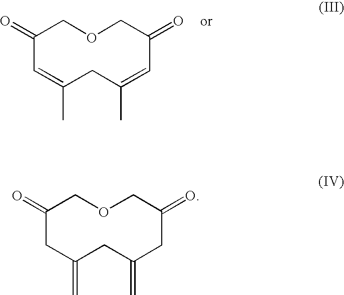

43. A compound with formula (I) or (II): 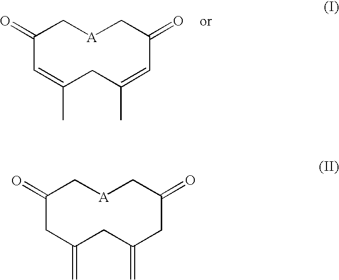
in which A is selected from NH, S, O, CH₂ and R—(CH₂)₂—R′ in which R and R′, independently of each other, are NH, O, S or CH₂, obtained by a method comprising:
a) culturing a bacterial strain; and
b) purifying the compound with formula (I) or (II) from the culture supernatant.

44. The compound according to claim 43, in which the bacterial strain is the JPL84 strain deposited at the CNCM with accession number CNCM I-3669.

45. The compound according to claim 43, which has the following formula:

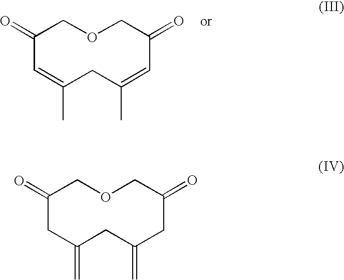

46. The compound according to claim 45, characterized in that the carbon in position 1 is a CH₂, the carbon in position 3 is a CH and the carbon in the terminal position is a CH₃.

47. The compound according to claim 43, which has a C₁₂/C₁₃ ratio which is different from the same compound produced chemically.

48. A composition comprising:
a) a compound with formula (I) or (II) or a mixture of said compounds: 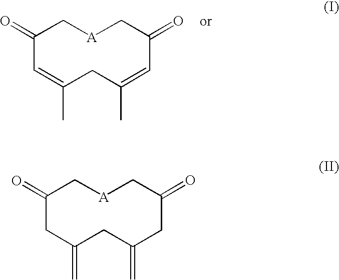
in which A is selected from NH, S, O, CH₂ and R—(CH₂)₂—R′ in which R and R′, independently of each other, are NH, O, S or CH₂; and
b) at least one pharmaceutically acceptable vehicle.

49. A composition comprising:
a) a compound with formula (I) or (II) or a mixture of said compounds: 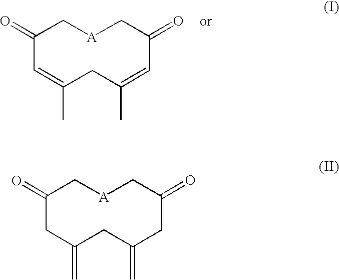
in which A is selected from NH, S, O, CH₂ and R—(CH₂)₂—R′ in which R and R′, independently of each other, are NH, O, S or CH₂; and
b) at least one second molecule which is active against micro-organisms, and optionally a pharmaceutically acceptable vehicle.

50. The composition according to claim 49, wherein the at least one second molecule which is active against micro-organisms is an antibiotic.

51. The composition according to claim 50, which comprises a compound with formula (I) and a compound with formula (II), in which A is identical in the two compounds and is selected from NH, S, CH₂ and R—(CH₂)₂—R′ in which R and R′, independently of each other, are NH, O, S or CH₂.

52. A drug with formula (I) or (II): 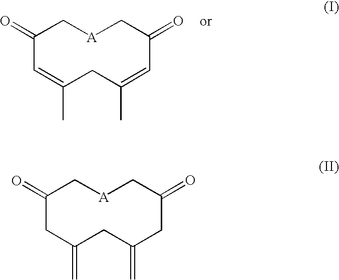
in which A is selected from NH, O, S, CH₂ and R—(CH₂)₂—R′ in which R and R′, independently of each other, are NH, O, S or CH₂.

53. An antibiotic or bactericide compound having the formula as defined in claim 52.

54. A drug composition comprising:
(a) a compound with formula (I) or (II) or a mixture of said compounds: 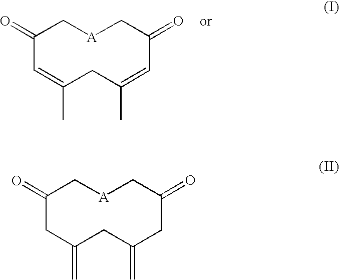
in which A is selected from NH, S, O, CH₂ and R—(CH₂)₂—R′ in which R and R′, independently of each other, are NH, O, S or CH₂; and
(b) at least one pharmaceutically acceptable vehicle and/or at least one second molecule which is active against micro-organisms.

55. An antibiotic or bactericide composition comprising:
(a) a compound with formula (I) or (II) or a mixture of said compounds: 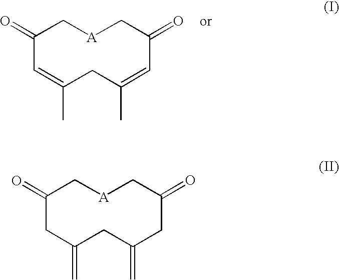
in which A is selected from NH, S, O, CH₂ and R—(CH₂)₂—R′ in which R and R′, independently of each other, are NH, O, S or CH₂; and
(b) at least one pharmaceutically acceptable vehicle and/or at least one second molecule which is active against micro-organisms.

56. A therapeutic or prophylactic method against bacterial infections, comprising the administration to a living being, of a compound with formula (I) or (II): 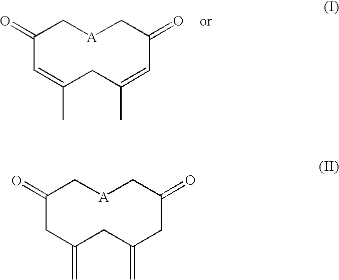
in which A is selected from NH, O, S, CH₂ and R—(CH₂)₂—R′ in which R and R′, independently of each other, are NH, O, S or CH₂ or of a composition comprising a compound with formula (I) or (II): 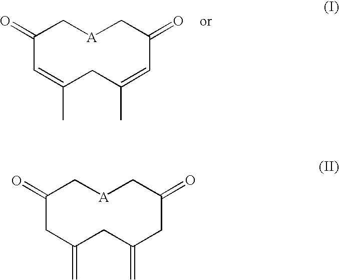
in which A is selected from NH, O, S, CH₂ and R—(CH₂)₂—R′ in which R and R′, independently of each other, are NH, O, S or CH₂.

57. The therapeutic or prophylactic method according to claim 56, wherein the bacterial infections are gram-positive bacterial infections.

58. A therapeutic or prophylactic method against Clostridium infections comprising the administration to a living being, of a compound or of a composition as defined in claim 56.

59. The therapeutic or prophylactic method according to claim 58, wherein the Clostridium infections are digestive infections linked to Clostridium difficile.

60. The therapeutic or prophylactic method according to claim 58, wherein the Clostridium infections are digestive infections linked to C. difficile consecutive to antibiotic therapy.

61. A method to restore the balance of the intestinal microbial flora of a patient comprising the administration to said living being of a compound or of a composition as defined in claim 56.

62. A method to manufacture impregnated devices comprising the impregnation of biomedical devices with a compound with formula (I) or (II): 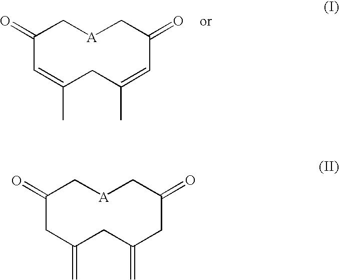
in which A is selected from NH, O, S, CH₂ and R—(CH₂)₂—R′ in which R and R′, independently of each other, are NH, O, S or CH₂ or with a composition comprising a compound with formula (I) or (II): 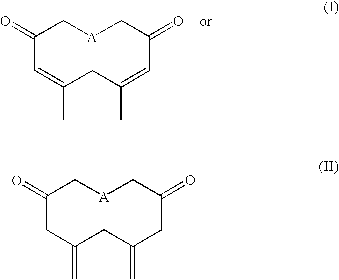
in which A is selected from NH, S, O, CH₂ and R—(CH₂)₂—R′ in which R and R′, independently of each other, are NH, O, S or CH₂.

63. The method according to claim 62, wherein the devices is a catheter, a dressing, a bone cement, a cerebral shunt or a cardiac valve.

64. The method according to claim 56, wherein the composition further comprises at least a pharmaceutically acceptable vehicle.

65. The method according to claim 56, wherein the composition further comprises at least one second molecule which is active against micro-organisms, and optionally a pharmaceutically acceptable vehicle.

66. The method according to claim 62, wherein the composition further comprises at least one second molecule which is active against micro-organisms.

67. The method according to claim 65, wherein the at least one second molecule which is active against micro-organisms is an antibiotic.

68. The method according to claim 56, wherein the composition comprises a compound with formula (I) and a compound with formula (II), in which A is identical in the two compounds and is selected from NH, S, CH₂ and R—(CH₂)₂—R′ in which R and R′, independently of each other, are NH, O, S or CH₂.

69. A biomedical device impregnated with a compound with formula (I) or 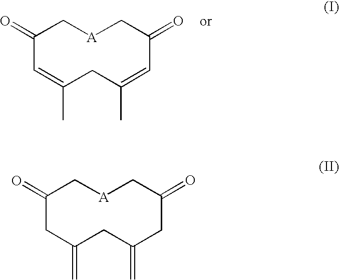
in which A is selected from NH, S, O, CH₂ and R—(CH₂)₂—R′ in which R and R′, independently of each other, are NH, O, S or CH₂ or with a composition comprising a compound with formula (I) or (II): 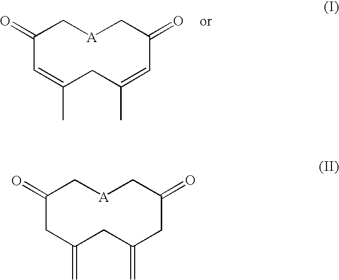
in which A is selected from NH, S, O, CH₂ and R—(CH₂)₂—R′ in which R and R′, independently of each other, are NH, O, S or CH₂.

70. The biomedical device according to claim 69, further impregnated with at least one second molecule which is active against micro-organisms.

71. The biomedical device according to claim 69, which is a catheter, a dressing, a bone cement, a cerebral shunt or a cardiac valve.

72. A bacterial strain which is capable of producing a compound with formula (I) or (II): 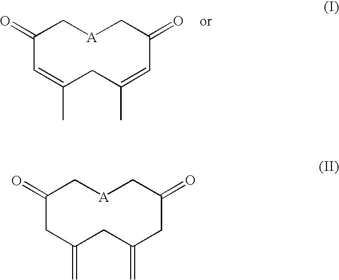
in which A is selected from NH, S, O, CH₂ and R—(CH₂)₂—R′ in which R and R′, independently of each other, are NH, O, S or CH₂.

73. The bacterial strain according to claim 72, which is the JPL84 strain deposited at the CNCM with accession number CNCM I-3669.

74. A strain derived from a bacterial strain according to claim 72, which has the capacity to produce a compound with formula (I) or (II).

75. The strain according to claim 72, which conserves the capacity to produce a compound with formula: 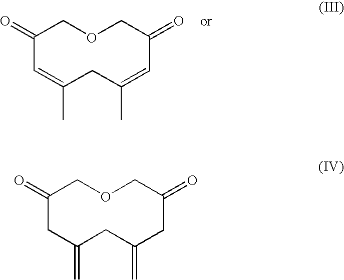
in which the carbon in position 1 is a CH₂, the carbon in position 3 is a CH and the carbon in the terminal position is a CH₃.

76. A method for modulating the bacterial profile of a biological sample or a surface, comprising bringing said sample or said surface into contact with at least one compound with formula (I) or (II) or a composition comprising compounds with formula (I) or (II), as defined in claim 56.
